(19) United States
(12) Patent Application Publication
(10) Pub. No.: US 2020/0237304 A1
(43) Pub. Date: Jul. 30, 2020

(54) HELMET

(71) Applicant: KYOCERA Corporation, Kyoto (JP)

(72) Inventor: Susumu KASHIWASE, Machida-shi, Tokyo (JP)

(73) Assignee: KYOCERA Corporation, Kyoto (JP)

(21) Appl. No.: 16/754,365

(22) PCT Filed: Oct. 1, 2018
(86) PCT No.: PCT/JP2018/036717

§ 371 Date: Apr. 7, 2020

(30) Foreign Application Priority Data
Oct. 12, 2017 (JP) 2017-198629

(51) Int. Cl. A61B 5/00; A61B 5/01; A61B 5/1455; A61B 5/026; A61B 5/0478; A61B 5/024; A61B 5/11

(52) U.S. Cl. CPC A61B 5/6803; A61B 5/0022; A61B 5/01; A61B 5/14552; A61B 2562/0219; A61B 5/0478; A61B 5/02438; A61B 5/1117; A61B 2560/0252; A61B 5/026

(57) ABSTRACT

A helmet includes a first sensor for detecting biological information of a wearer, a second sensor for detecting environment information around the wearer, and a controller configured to acquire the biological information and the environment information from the first sensor and the second sensor, respectively. The controller determines a cycle for executing detection by at least one of the first sensor and the second sensor, based on the biological information and the environment information.

### CROSS REFERENCE TO RELATED APPLICATION

This application claims priority to and the benefit of Japanese Patent Application No. 2017-198629 filed on Oct. 12, 2017, the entire contents of which are incorporated herein by reference.

### TECHNICAL FIELD

The present disclosure relates to a helmet.

### BACKGROUND

A helmet equipped with a sensor device which is capable of detecting biological information, such as body temperature or heart rate, of a wearer of the helmet is known (e.g., see PTL 1).

### CITATION LIST

### Patent Literature

PTL 1: JP-A-2010-148718

### SUMMARY

A helmet according to an embodiment of the present disclosure includes a first sensor for detecting biological information of a wearer, and a second sensor for detecting environment information around the wearer. The helmet includes a controller configured to acquire the biological information and the environment information from the first sensor and the second sensor, respectively. The controller determines a cycle for executing detection by at least one of the first sensor and the second sensor, based on the biological information and the environment information.

### BRIEF DESCRIPTION OF THE DRAWINGS

In the accompanying drawings:

FIG. 1 is a side view schematically illustrating an example configuration of a helmet according to an embodiment;

FIG. 2 is a block diagram schematically illustrating an example configuration of the helmet according to the embodiment;

FIG. 3 is a table illustrating an example relationship between the temperature and the relative humidity;

FIG. 4 is a flowchart illustrating an example procedure performed by a controller of the helmet;

FIG. 5 is a block diagram illustrating an example configuration in which the helmet and a server are connected to each other;

FIG. 6 is a flowchart illustrating an example including a procedure for transmitting biological information or environment information to the server;

FIG. 7 is a flowchart illustrating an example procedure of a communication operation subroutine;

FIG. 8 is a flowchart illustrating an example procedure executed by the server; and

FIG. 9 is a flowchart illustrating an example procedure for detecting a change in a physical condition of a wearer.

### DETAILED DESCRIPTION

A helmet can be equipped with a sensor device for detecting biological information of a wearer of the helmet, such as body temperature or the heart rate. Battery capacity may be limited in order to reduce the weight and size of the sensor device. Even when battery capacity is limited, it is desired to extend the time period for which the sensor device can detect the biological information. It is also desired to accurately determine a physical condition of the wearer of the helmet, based on the biological information detected by the sensor device.

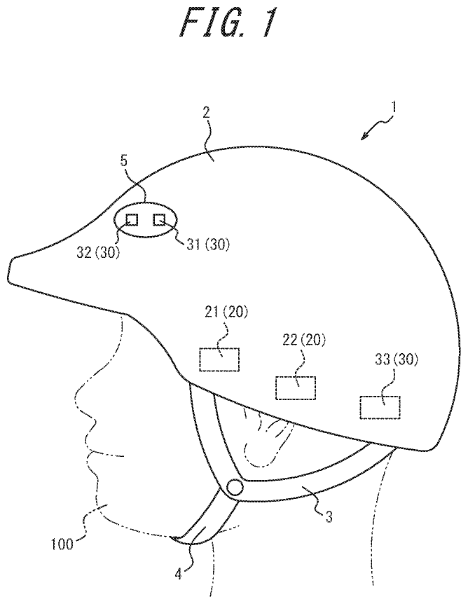

A helmet 1 according to an embodiment includes a cap body 2, an ear strap 3, and a chin strap 4, as illustrated in FIG. 1. The helmet 1 can be worn by a wearer 100, which is represented by the virtual two-dot chain lines.

The helmet 1 further includes a first sensor 20 and a second sensor 30. Each of the first sensor 20 and the second sensor 30 may be arranged on the cap body 2, the ear strap 3, or the chin strap 4.

The first sensor 20 may be a biological sensor for acquiring biological information from the wearer 100. The first sensor 20 may include a pulse sensor 21 for detecting the pulse of the wearer 100. The pulse sensor 21 may detect the pulse rate of wearer 100 by detecting the blood flow through the artery of the wearer 100 based on the reflected light of the light irradiated to the artery of the wearer 100. The pulse sensor 21 may detect the pulse rate of the wearer 100 by detecting movement of the artery or the skin in the vicinity of the artery of the wearer 100 as an acceleration. The detection method of the pulse sensor 21 is not limited thereto, and the pulse sensor 21 can detect the pulse rate of the wearer 100 in various manners.

The first sensor 20 may include a body temperature sensor 22 for detecting the body temperature of the wearer 100. The body temperature sensor 22 may be configured as a temperature sensor of various types, such as a thermistor. The body temperature sensor 22 may come into contact with the forehead or the temple of the wearer 100. The body temperature sensor 22 may come into contact with the body surface in the vicinity of the artery of the wearer 100.

The first sensor 20 may include a blood flow sensor for detecting the blood flow of the wearer 100. The blood flow sensor may detect the blood flow through the artery of the wearer 100, based on the reflected light of the light irradiated to the artery of the wearer 100. The blood flow sensor may detect the blood flow in other various manners.

The first sensor 20 may include an oxygen saturation sensor for detecting an oxygen saturation in the blood of the wearer 100. The oxygen saturation sensor may detect, for example, a percutaneous arterial oxygen saturation. The percutaneous arterial oxygen saturation is also referred to as SpO₂. Here, “S” represents the saturation, “P” represents percutaneous or pulse oximetry, and “O₂” represents oxygen. Hereinafter, the percutaneous arterial oxygen saturation will also be simply referred to as the oxygen saturation. The oxygen saturation may be represented by a measured value of the oxygen saturation of the arterial blood. The oxygen saturation sensor may detect the oxygen saturation of the arterial blood, based on the reflected light of the light irradiated to the artery of the wearer 100. The oxygen saturation of the arterial blood is also referred to as SaO₂. Here, “a” represents the artery. SpO₂ can be considered to be a method of indirectly measuring SaO₂. Under regulated measurement conditions, SpO₂ can provide an approximate value of SaO₂.

The first sensor 20 may include a brain wave sensor for detecting brain waves of the wearer 100. The brain wave sensor may detect brain waves by detecting, for example, a change in a voltage via an electrode in contact with the body surface of the wearer 100.

The first sensor 20 may be configured as one sensor having a plurality of different functions. For example, the first sensor 20 may be configured as one sensor having a function of the pulse sensor 21, a function of the oxygen saturation sensor, and a function of the blood flow sensor. The first sensor 20 may detect various biological information other than the pulse, the oxygen saturation, the blood flow, and the body temperature. The first sensor 20 may be configured as one sensor having an appropriate combination of functions for detecting various biological information.

The first sensor 20 may be arranged in the vicinity of the artery of the wearer 100. The first sensor 20 may be arranged in the vicinity of the temple of the wearer 100. The first sensor 20 may be in contact with the body surface of the wearer 100, or may be arranged at a position spaced apart from the body surface by a predetermined distance. The first sensor 20 is not limited to the above examples and may be arranged in various manners capable of detecting the biological information of the wearer 100.

The second sensor 30 may be an environment sensor for obtaining environment information around the wearer 100. The second sensor 30 may include an ambient temperature sensor 31 for detecting the temperature around the wearer 100. The ambient temperature sensor 31 may be configured as a temperature sensor of various types, including a thermistor. The second sensor 30 may include a humidity sensor 32 for detecting a relative humidity of the ambient air around the wearer 100. The humidity sensor 32 may be configured as a sensor for detecting a relative humidity based on a dry-bulb temperature and a wet-bulb globe temperature, a sensor that uses a hygroscopic material, or a sensor of various other types.

The second sensor 30 may include a motion sensor 33 for detecting movement, orientation, or the like of the helmet 1. The motion sensor 33 may be configured as a six-axis sensor capable of detecting acceleration in three axes in translational directions and angular velocity in three axes in rotational directions. It can be said that the second sensor 30 detects the motion, the posture, or the like of the wearer 100 as the environment information using the motion sensor 33. The motion sensor 33 may be included in the first sensor 20. In this case, it can be said that the first sensor 20 detects the motion, the posture, or the like of the wearer 100 as the biological information using the motion sensor 33. A controller 10 can determine whether the wearer 100 is in an abnormal state such as stumbling or having fallen, based on a detection result by the motion sensor 33.

The second sensor 30 may include a positioning sensor for detecting a position of the helmet 1. The positioning sensor may acquire position information of the helmet 1 based on, for example, GPS (Global Positioning System) or GNSS (Global Navigation Satellite System).

The cap body 2 has a first surface facing outward and a second surface facing the wearer 100. The cap body 2 may have a hole 5 penetrating from the first surface to the second surface. The hole 5 can be regarded as penetrating between the wearer 100 and the exterior. Ambient air can be taken into the cap body 2 through the hole 5. The ambient temperature sensor 31 may be arranged at the hole 5. The ambient temperature sensor 31 may be arranged inside the hole 5. The ambient temperature sensor 31 may be arranged inside the hole 5 or the cap body 2 in such a manner as to be visible from the exterior of the cap body 2. The ambient temperature sensor 31 may be arranged at a position easily exposed to the ambient air. In this way, when the ambient temperature sensor 31 detects the ambient temperature of the wearer 100, the temperature detected by the ambient temperature sensor 31 is less affected by the body heat of the wearer 100. The ambient temperature sensor 31 may be arranged at a position that is not exposed to direct sunlight. In this way, the temperature detected by the ambient temperature sensor 31 is less affected by direct sunlight.

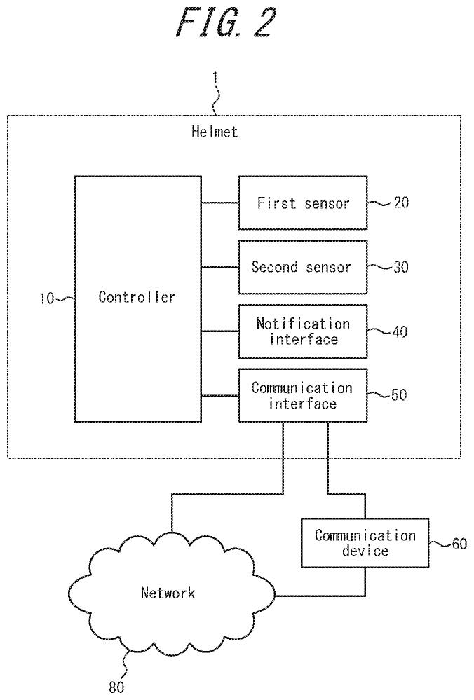

The helmet 1 includes the controller 10, the first sensor 20, and the second sensor 30, as illustrated in FIG. 2. The helmet 1 may further include a notification interface 40. The helmet 1 may further include a communication interface 50.

The controller 10 can control and manage each element of the helmet 1. The controller 10 may be configured to include at least one processor such as a CPU (Central Processing Unit) configured to execute a program defining a control procedure. The controller 10 may include a memory for storing a program. The controller 10 may be connected to an external storage medium that stores a program.

At least one processor may be configured to include one integrated circuit (IC), or a plurality of ICs or discrete circuits communicatively connected to one another. At least one processor may be configured according to various known techniques. The processor may be configured to include, for example, one or more circuits or units configured to execute one or more processing according to instructions stored in the memory or the storage medium. The processor may be configured as firmware configured to execute one or more processing. The firmware may be, for example, a discrete logic component.

The processor may be configured to include one or more processors, controllers, microprocessors, microcontrollers, application specific ICs, digital signal processors, programmable logic devices, field programmable gate arrays, or the like. The processor may be configured as any appropriate combination of these devices or configurations thereof, or any appropriate combination of other known devices or configurations thereof.

The notification interface 40 notifies the user of information based on a control instruction acquired from the controller 10. The notification interface 40 may include a display device. The display device may be configured as, for example, a liquid crystal display, an organic EL (Electroluminescent) display, or an inorganic electroluminescent display, but is not limited thereto. The notification interface 40 may notify the information to the wearer 100 or a person located near the wearer 100 by displaying characters or an image on the display device, based on the control instruction acquired from the controller 10.

The notification interface 40 may include a light source such as an LED (Light Emitting Diode) or a halogen lamp. The notification interface 40 may notify information to the wearer 100 or a person located near the wearer 100 by turning on or off the light source, based on the control instruction acquired from the controller 10. The notification interface 40 may include a buzzer such as a piezoelectric buzzer or a solenoid buzzer, or a speaker for generating a predetermined sound. The notification interface 40 may notify information to the wearer 100 or a person located near the wearer 100 by sounding a buzzer or generating a sound from the speaker, based on the control instruction acquired from the controller 10.

The communication interface 50 may include, for example, a communication interface such as a LAN (Local Area Network). The communication interface 50 may be communicably connected to a communication device 60, which is externally provided, in a wired or wireless manner via the communication interface. The communication interface 50 may be communicatively connected to a network 80 via the communication device 60. The communication interface 50 may be communicatively connected to the network 80 in a direct manner. The communication device 60 may include a positioning sensor. The controller 10 may acquire position information detected by the positioning sensor from the communication device 60.

The controller 10 may acquire the biological information of the wearer 100 and the environment information around the wearer 100 from the first sensor 20 and the second sensor 30, respectively. The biological information and the environment information are collectively referred to as sensor information. The controller 10 may determine whether there is a change in the physical condition of the wearer 100, based on at least some of the sensor information. The controller 10 may calculate a heat index, based on the environment information. The heat index is also referred to as WBGT (Wet-Bulb Globe Temperature). The controller 10 may determine whether there is a change in the physical condition of the wearer 100, based on the WBGT and the biological information of the wearer 100. The controller 10 may calculate, for example, the probability that the wearer 100 is suffering from heat stroke, or determine whether there are signs that the wearer 100 will suffer from heat stroke.

The WBGT can be calculated based on the dry-bulb temperature (T_{D}), the wet-bulb globe temperature (T_{W}), and the black-bulb temperature (T_{G}). The WBGT may be calculated using Equation (1) and Equation (2) set forth below, by way of example. Degrees ° C. will be used as units of the dry-bulb temperature, wet-bulb globe temperature, and black-bulb temperature.

Under outdoor condition with sunlight,

WBGT (degrees)=0.7×T_{W}+0.2×T_{G}+0.1×T_{D} (1)

is satisfied.

Under indoor condition without sunlight,

WBGT (degrees)=0.7×T_{W}+0.3×T_{G} (2)

is satisfied.

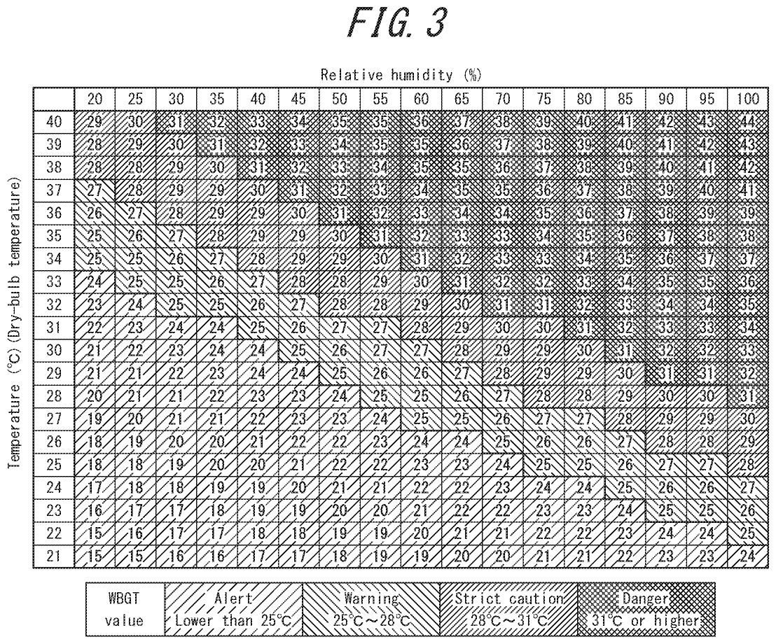

The WBGT can be calculated based on, for example, the relationship between the temperature and the relative humidity as indicated in the table (referenced from “Heat Stroke Prevention Guidance in Daily Life” by the Japanese Society of Biometeorology) shown in FIG. 3 by way of example. The temperature may be dry-bulb temperature. For example, when the temperature is 30° C. and, simultaneously, the relative humidity is 70%, the WBGT may be set as 29 degrees. The larger the value of the WBGT, the higher the probability that the person suffers from heat stroke. In the table illustrated in FIG. 3, the values of the WBGT are classified into categories of lower than 25 degrees, 25 degrees or higher and lower than 28 degrees, 28 degrees or higher and lower than 31 degrees, and 31 degrees or higher. Each cell in which a value of WBGT is indicated is distinguished by hatching according to the respective category of the value. The cells in which the WBGT is lower than 25 degrees are hatched by upward diagonal lines. The cells in which the WBGT is 25 degrees or higher and lower than 28 degrees are hatched by downward diagonal lines. The cells in which the WBGT is 28 degrees or higher and lower than 31 degrees are hatched by upward diagonal lines that are arranged at narrower intervals than those in the cells in which the WBGT is lower than 25 degrees. The cells in which the WBGT is 31 degrees or higher are hatched with upward and downward diagonal lines crossing one another. When the value of the WBGT is lower than 25 degrees, an alert for heat stroke may be issued. When the value of the WBGT is 25 degrees or higher and lower than 28 degrees, a warning against heat stroke may be issued. When the value of the WBGT is 28 degrees or higher and lower than 31 degrees, strict caution against heat stroke may be prompted. When the value of the WBGT is 31 degrees or higher, a person can be considered to be at risk of developing heat stroke.

The controller 10 may calculate the probabilities, or determine whether there are signs, that the wearer 100 may develop various sicknesses other than heat stroke. The controller 10 may determine whether the wearer 100 is asleep, or there are signs that the wearer 100 may fall asleep, based on the biological information and the environment information of the wearer 100.

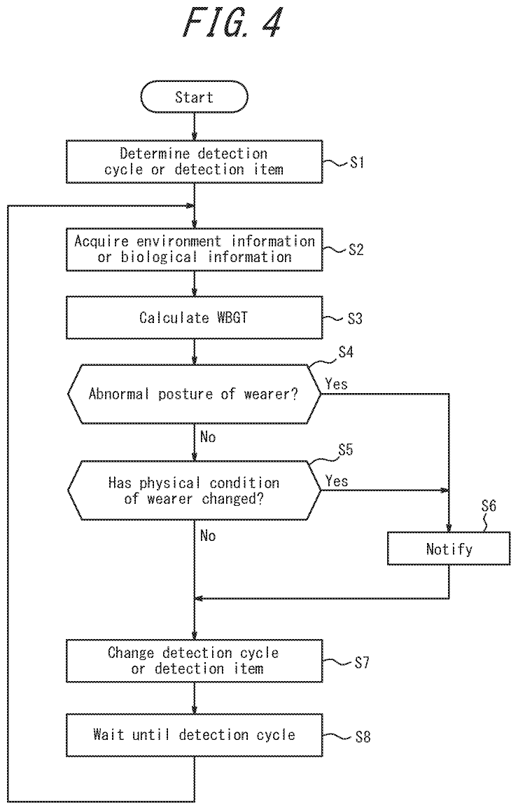

The controller 10 may determine whether there is a change in the physical condition of the wearer 100 using the procedure in the flowchart illustrated in FIG. 4.

The controller 10 sets a cycle for detecting the sensor information by at least one of the first sensor 20 and the second sensor 30, or sets items to be detected as the sensor information (step S1). The cycle for detection of the sensor information may also be referred to as a detection cycle. The items to be detected as the sensor information may also be referred to as a detection item. The detection cycle may be set in, for example, seconds, minutes, or more than 1 hour as a unit. The detection cycle may be set to vary for each detection item. The detection items may include, for example, the body temperature or the heart rate of the wearer 100, or the temperature or the humidity around the wearer 100. In a case in which the detection cycle is short or there are many detection items, the state of the wearer 100 can be accurately recognized In a case in which the detection cycle is long or there are a small number of detection items, power consumption by the controller 10, the first sensor 20, and the second sensor 30 can be reduced.

The controller 10 acquires at least one of the environment information around the wearer 100 and the biological information of the wearer 100, based on the detection cycle and the detection item (step S2). The environment information may include the temperature or the relative humidity around the wearer 100. The biological information may include the body temperature or the heart rate of the wearer 100. The environment information or the biological information may include information indicating a motion or an orientation of the helmet 1. The controller 10 may acquire the environment information from the second sensor 30 and the biological information from the first sensor 20 at the detection cycle of the information by the second sensor 30.

The controller 10 calculates the WBGT based on the environment information (step S3). In a case in which the environment information includes the temperature and the relative humidity around the wearer 100, the controller 10 may calculate the WBGT based on, for example, the table illustrated in FIG. 3. In a case in which the environment information includes the temperature, the black bulb temperature, and the wet-bulb globe temperature, the controller 10 may calculate the WBGT based on Equation (1) or Equation (2) set forth above.

In a case in which the biological information or the environment information includes motion or orientation of the helmet 1, the controller 10 determines whether a posture of the wearer 100 is abnormal (step S4). The motion of the helmet 1 can represent the motion of the head of the wearer 100. When the controller 10 determines that the head of the wearer 100 is unsteady or motionless, or that the wearer 100 has fallen over based on the motion of the helmet 1, the controller 10 may determine that the posture of the wearer 100 is abnormal. The controller 10 may determine that the posture of the wearer 100 is abnormal, based on various motions of the helmet 1 other than the above motions. When the controller 10 determines that the posture of the wearer 100 is abnormal (Yes in step S4), the controller 10 proceeds to step S6.

When the controller 10 determines that the posture of the wearer 100 is not abnormal (No in step S4), the controller 10 determines whether there is, or there are signs of, a change in the physical condition of the wearer 100, based on the WBGT and the biological information of the wearer 100 (step S5). For example, when the body temperature of the wearer 100 exceeds a predetermined threshold, the controller 10 may determine that the wearer 100 has developed heat stroke, or that there are signs of developing heat stroke. For example, when the heart rate of the wearer 100 exceeds a predetermined threshold, the controller 10 may determine that the wearer 100 has developed heat stroke, or that there are signs of developing heat stroke. The controller 10 may determine whether there is, or there are signs of, a change in the physical condition of the wearer 100, based on various other information included in the sensor information. When controller 10 determines that there is no change in the physical condition of the wearer 100 and, simultaneously, there are no signs of a change (No in step S5), the controller 10 proceeds to step S7.

When the controller 10 determines that the posture of the wearer 100 is abnormal (Yes in step S4), or determines that there is, or there are signs of, a change in the physical condition of the wearer 100 (Yes in step S5), the controller 10 causes the notification interface 40 to notify accordingly (step S6). The controller 10 may output control information including the notification content to the notification interface 40. The notification interface 40 may notify information for calling the attention of the wearer 100 or the surroundings to the wearer 100 or a person located near the wearer 100. The information for calling the attention of the wearer 100 or the surroundings is also referred to as alert information. The alert information may include information regarding techniques for coping with heat stroke or techniques for preventing heat stroke. After completing step S6, the controller 10 proceeds to step S7.

The controller 10 changes the detection cycle or the detection item based on the WBGT (step S7). When the WBGT is, for example, 31 degrees or higher indicating that there is a risk that a human may develop heat stroke, the controller 10 may reduce the detection cycle or increase the number of detection items. When the WBGT is, for example, lower than 21 degrees indicating that a human is substantially free from a risk of developing heat stroke, the controller 10 may increase the detection cycle or reduce the number of detection items. The controller 10 may change the detection cycle or the detection item, based on a result of a comparison of the WBGT to various other values.

The controller 10 uses a timer and waits for the detection cycle that has been set (step S8). The controller 10 may return to step S2 when the detection cycle has elapsed.

The helmet 1 according to the present embodiment can change the detection cycle or the detection item while monitoring a change, or signs of a change, in the physical condition of the wearer 100. By virtue of the increase in the detection cycle or the reduction in the number of detection items performed by the controller 10 based on the WBGT, the controller 10, the first sensor 20, or the second sensor 30 can be activated for a longer period within a predetermined battery capacity. By virtue of the reduction in the detection cycle or the increase in the number of detection items performed by the controller 10 based on the WBGT, whether there is a change or signs of a change in the physical condition of the wearer 100 can be accurately determined. According to the helmet 1 of the present embodiment, that is, the sensor information can be detected for a longer period within the predetermined battery capacity, and a change in the physical condition of the wearer 100 can be accurately determined.

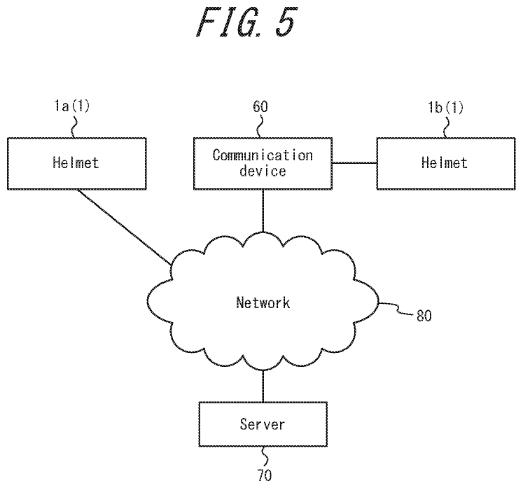

Each of a helmet la and a helmet lb may be communicably connected to a server 70 via the network 80, as illustrated in FIG. 5. The network 80 may be communicably connected to the helmet la in a direct manner. The network 80 may be communicably connected to the helmet lb via the communication device 60. The network 80 may be communicably connected to the server 70 in a wired or wireless manner. The helmet 1a and the helmet 1b are collectively referred to as the helmet 1.

The server 70 may acquire at least one of the biological information of the wearer 100 and the environment information around the wearer 100 from the helmet 1. The server 70 may determine whether there is a change in the physical condition of the wearer 100, based on at least a part of the sensor information acquired from the helmet 1. The server 70 may transmit information regarding a result of the determination as to whether there is a change in the physical condition of the wearer 100 to the helmet 1. The server 70 may transmit the alert information to the helmet 1 when the server 70 determines that there is, or there are signs of, a change in the physical condition of the wearer 100. The server 70 may determine the detection cycle or the detection item of the helmet 1. The server 70 may differentiate or match the detection cycles of the helmets 1a and 1b or the detection items of the helmets 1a and 1b. The server 70 may transmit information regarding the detection cycle or the detection item of the helmet 1 to the helmet 1. Information regarding a change in the detection cycle or the detection item will also be referred to as change information.

The server 70 may correct the environment information, based on the environment information acquired from a plurality of helmets 1. The server 70 may analyze the environment information acquired from a plurality of helmets 1 by performing various operations such as, for example, averaging. The server 70 may analyze the environment information using a statistical method. The server 70 may determine whether the helmet 1 is located within a predetermined range, based on the position information from the positioning sensor. The server 70 may analyze the environment information acquired from the helmet 1 located within the predetermined range. The server 70 may correct the environment information, based on a result of analysis on the environment information acquired from a plurality of helmets 1. The server 70 may estimate the physical condition of the wearer 100 or determine the detection cycle or the detection item of the helmet 1, based on corrected environment information.

The server 70 may acquire the result of determination on whether there is a change in the physical condition of the wearer 100 from the helmet 1. The server 70 may analyze a result of the determination as to whether there is a change in the physical condition of the wearer 100 acquired from each of a plurality of helmets 1 by performing various operations or using a statistical method. The server 70 may acquire and analyze a result of a determination as to whether there is a change in the physical condition of the wearer 100 located within a predetermined range. The server 70 may change the threshold used for the determination that there is a change in the physical condition of the wearer 100 located within the predetermined location, based on the result of statistical analysis on the result of the determination as to whether there is a change in the physical condition of the wearer 100. For example, for wearers 100 located within the predetermined range, when it is determined that a large proportion of the wearers 100 have a change in their physical condition, the server 70 may determine that an event which affects the detected value from the first sensor 20 or the second sensor 30 has occurred within the predetermined range. In this case, the server 70 may change the threshold used for the determination on that there is a change in the physical condition of the wearer 100 to a value that facilitates recognition of a change, or a value that impedes recognition of a change.

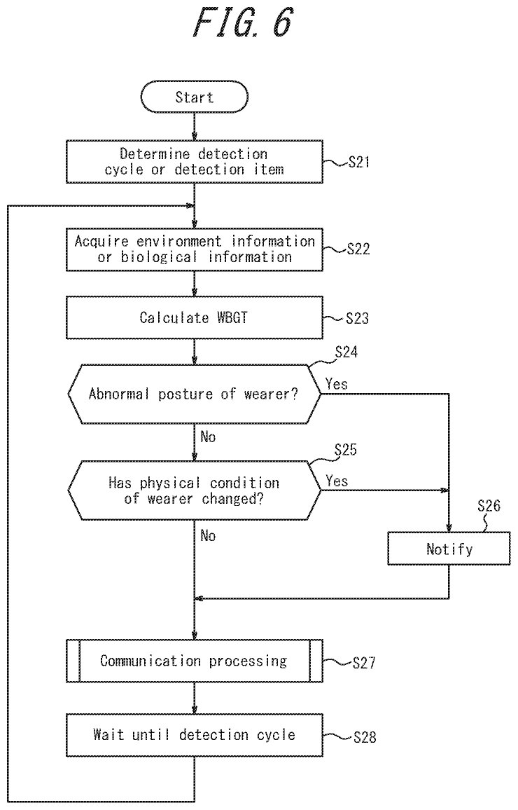

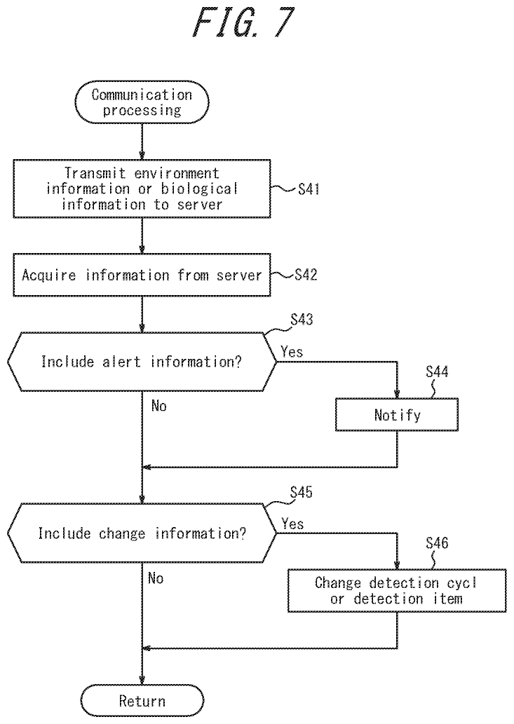

The controller 10 may determine whether there is a change in the physical condition of the wearer 100 using the procedures of the flowcharts illustrated in FIG. 6 and FIG. 7. Each of the flowcharts illustrated in FIG. 6 and FIG. 7 differs from the flowchart illustrated in FIG. 4, in terms of inclusion of a procedure regarding the communication processing with respect to the network 80. The communication processing between the helmet 1 and the network 80 may be executed following the procedures specified in step S27 of FIG. 6 and each step of FIG. 7.

In the procedure from step S21 to S26 of FIG. 6, the controller 10 executes processing which is the same as, or similar to, the processing from step S1 to S6 of FIG. 4. The controller 10 determines whether the posture of the wearer 100 is abnormal in step S24, and whether there is, or there are signs of, a change in the physical condition of the wearer 100 in step S25. Then, the controller 10 proceeds to step S27.

The controller 10 executes the communication processing subroutine (step S27). The controller 10 executes the communication processing using the procedures of the flowchart illustrated in FIG. 7.

The controller 10 transmits the environment information or the biological information to the server 70 via the network 80 (step S41). The environment information may include the temperature or the relative humidity around the wearer 100. The biological information may include the body temperature or the heart rate of the wearer 100. The environment information or the biological information may include information indicating a motion or an orientation of the helmet 1.

The server 70 may determine whether there is, or there are signs of, a change in the physical condition of the wearer 100 of the helmet 1, or determine to change the detection cycle or the detection item, based on the sensor information acquired from the helmet 1. When the server 70 determines that there is, or there are signs of, a change in the physical condition of the wearer 100, the server 70 may transmit the alert information for alerting the wearer 100 or people around the wearer 100 to the helmet 1. When the server 70 determines to change the detection cycle or the detection item, the server 70 may transmit the change information to the helmet 1.

The controller 10 acquires the information from the server 70 (step S42). The information acquired from the server 70 may include the alert information or the change information.

The controller 10 determines whether the information acquired from the server 70 includes the alert information (step S43). When the information acquired from the server 70 does not include the alert information (No in step S43), the controller 10 proceeds to step S45.

When the information acquired from the server 70 includes the alert information (Yes in step S43), the controller 10 causes the notification interface 40 to notify accordingly, in accordance with the alert information (step S44). The controller 10 may execute processing the same as, or similar to, the processing in step S6 of the flowchart illustrated in FIG. 4. After completing step S44, the controller 10 proceeds to step S45.

The controller 10 determines whether the information acquired from the server 70 includes the change information (step S45). When the information acquired from the server 70 does not include the change information (No in step S45), the controller 10 ends the communication processing subroutine following the procedure of the flowchart illustrated in FIG. 7 and returns to step S28 of FIG. 6.

When the information acquired from the server 70 includes the change information (Yes in step S45), the controller 10 changes the detection cycle or the detection item, based on the change information (step S46). After completing step S46, the controller 10 ends the communication processing subroutine following the procedure of the flowchart illustrated in FIG. 7 and returns to step S28 of FIG. 6.

The controller 10 uses the timer and waits for the detection cycle that has been set (step S28 of FIG. 6). When the detection cycle has elapsed, the controller 10 returns to step S22 of FIG. 6.

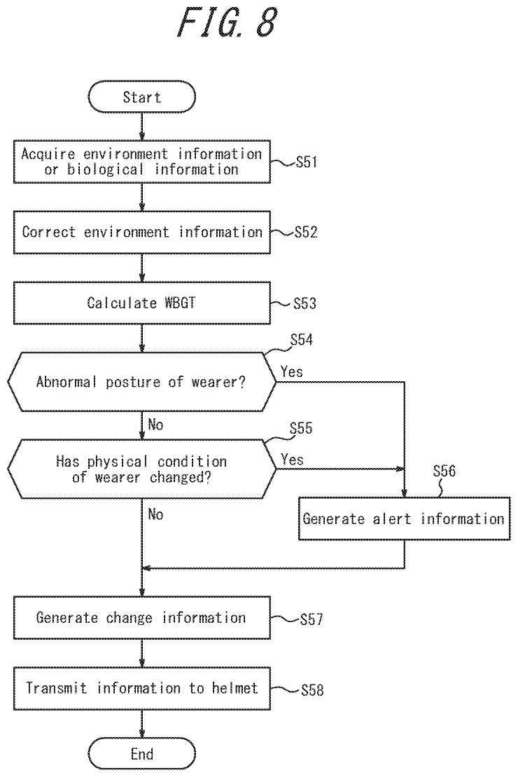

The server 70 may generate the information to be transmitted to the helmet 1, following the procedure of the flowchart illustrated in FIG. 8.

The server 70 acquires the environment information or the biological information from the helmet 1 (step S51). The environment information may include the temperature or the relative humidity around the wearer 100. The biological information may include the body temperature or the heart rate of the wearer 100. The environment information or the biological information may include the information indicating motion or orientation of the helmet 1.

When the server 70 receives the environment information from a plurality of helmets 1, the server 70 corrects the environment information, based on the environment information acquired from each of the helmets 1 (step S52). The server 70 may statistically process the environment information performing, for example, averaging. The server 70 may determine whether the environment information acquired from each of the helmets 1 includes an error, based on a result of the statistical processing. The server 70 may correct an error included in the environment information. In this way, an erroneous detection by the second sensor 30 can be corrected. As a result, the accuracy in the determination on whether there is a change in the physical condition of the wearer 100 of the helmet 1 may be improved.

The server 70 calculates the WBGT based on the environment information. The server 70 may perform processing the same as, or similar to, the processing of step S2 of FIG. 4. The server 70 may calculate the WBGT based on corrected environment information.

The server 70 determines whether the posture of the wearer 100 is abnormal (step S54). The server 70 may determine in the same manner as, or in a manner similar to, the processing of step S4 of FIG. 4. When the posture of the wearer 100 is not abnormal (No in step S54), the server 70 determines whether there is, or there are signs of, a change in the physical condition of the wearer 100 (step S55). The server 70 may determine in the same manner as, or in a manner similar to, the processing of step S5 of FIG. 4. When the server 70 determines that there is not, or there are no signs of, a change in the physical condition of the wearer 100, the server 70 proceeds to step S57.

When the posture of the wearer 100 is abnormal (Yes in step S54), or when there is, or there are signs of, a change in the physical condition of the wearer 100 (Yes in step S55), the server 70 generates the alert information (step S56). The alert information may include the content to be notified by the notification interface 40 of the helmet 1. After completing step S56, the controller 10 proceeds to step S57.

The server 70 generates the change information for changing the detection cycle or the detection item of the helmet 1, based on the WBGT (step S57). The server 70 may generate the change information based on the determination the same as, or similar to, the processing in step S7 of FIG. 4. The server 70 may generate the change information based on the WBGT calculated from the corrected environment information.

The server 70 transmits the information including the alert information or the change information to the helmet 1 (step S58). After completing step S58, the server 70 ends the procedure of the flowchart illustrated in FIG. 8.

The helmet 1 according to the present embodiment transmits the environment information or the biological information to the server 70 via the network 80 and acquires information from the server 70. The information transmitted from a plurality of helmets 1 may be analyzed using various operations or statistical methods by the server 70. The determination based on the result of the analysis of the information transmitted from a plurality of the helmets 1 enables close monitoring of a change, or signs of a change, in the physical condition of the wearer 100 of each of the helmets 1.

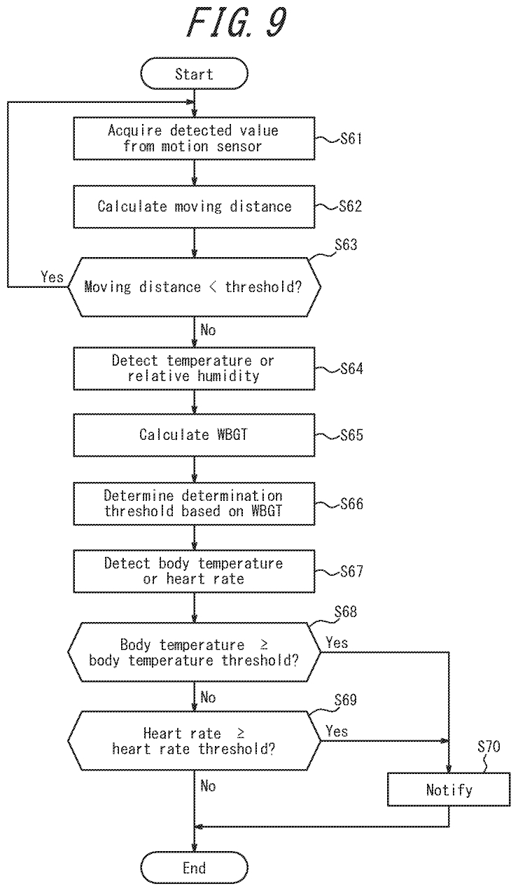

The helmet 1 according to an embodiment can determine whether there is, or there are signs of, a change in the physical condition of the wearer 100, in accordance with the procedure of the flowchart illustrated in FIG. 9.

The controller 10 acquires a detected value from the motion sensor 33 (step S61). The detected value from the motion sensor 33 may be acquired as the environment information or the biological information. The detection value by the motion sensor 33 may include acceleration in the three axes in translational directions or angular velocity in the three axes in rotational directions.

The controller 10 calculates a moving distance of the wearer 100 within a predetermined period, based on the detected value from the motion sensor 33 (step S62). The controller 10 may calculate the moving distance of the wearer 100 by integrating the acceleration values within the predetermined time period. In a case in which the wearer 100 is riding on a vehicle such as a motorcycle or a bicycle, the moving distance of the wearer 100 may be a running distance of the vehicle. The predetermined period may be the detection cycle or a time period determined by another criterion. The controller 10 may calculate the moving distance of the wearer 100 within the predetermined time period, based on a detected value from the positioning sensor.

The controller 10 determines whether the moving distance within the predetermined time period is smaller than a threshold (step S63). When the moving distance within the predetermined time period is smaller than the threshold (Yes in step S63), the controller 10 returns to step S61.

When the moving distance within the predetermined time period is equal to or larger than the threshold (No in step S63), the controller 10 acquires a value of the temperature detected by the ambient temperature sensor 31 or a value of the relative humidity detected by the humidity sensor 32 (step S64). When the moving distance within the predetermined time period is equal to or larger than the threshold, it can be said that the moving speed is equal to or faster than a predetermined speed.

When the ambient temperature sensor 31 and the humidity sensor 32 are located at the holes 5 of the helmet 1, ventilation through the holes 5 enables these sensors to detect a temperature or a relative humidity which is less affected by the body temperature or perspiration of the wearer 100. When the moving speed is equal to or faster than the predetermined speed, the holes 5 can ventilate more. That is, when the moving speed is equal to or faster than the predetermined speed, the ambient temperature sensor 31 and the humidity sensor 32 located at the holes 5 can more accurately detect the temperature or the relative humidity.

On the other hand, when the moving speed is less than the predetermined speed, there is a probability that the holes 5 do not sufficiently ventilate the helmet 1. In this case, the temperature detected by the ambient temperature sensor 31 and the relative humidity detected by the humidity sensor 32 are more affected by the body temperature or perspiration of the wearer 100. That is, the detected value of the temperature or the relative humidity when the moving speed is less than the predetermined speed may include a large error. By refraining from making a determination based on the value of the temperature or the relative humidity that may include an error, an erroneous determination can be avoided.

The controller 10 calculates the WBGT (step S65). The controller 10 may calculate the WBGT by executing the processing the same as, or similar to, the processing of step S3 of FIG. 4.

The controller 10 determines the threshold to be used for the determination as to whether the wearer 100 has developed heat stroke, or whether there are signs that the wearer 100 may develop heat stroke, based on the WBGT (step S66). The controller 10 may determine a body temperature threshold to be compared to the body temperature of the wearer 100. When the body temperature of the wearer 100 is equal to or higher than the body temperature threshold, the controller 10 may determine that the wearer 100 has developed heat stroke, or that there are signs that the wearer 100 may develop heat stroke. When the WBGT is lower than 24 degrees, the controller 10 may determine the body temperature threshold to be, for example, 37° C. When the WBGT is 24 degrees or higher and lower than 31 degrees, the controller 10 may determine the body temperature threshold to be, for example, 38° C. When the WBGT is 31 degrees or higher, the controller 10 may determine the body temperature threshold to be, for example, 39° C. That is, the controller 10 may determine the body temperature threshold to be higher, as the WBGT becomes higher.

The controller 10 may determine a heart rate threshold to be compared to the heart rate of the wearer 100. When the heart rate of the wearer 100 is equal to or higher than the heart rate threshold, the controller 10 may determine that the wearer 100 has developed heat stroke, or that there are signs that the wearer 100 may develop heat stroke. The controller 10 may acquire the heart rate threshold by multiplying a difference between a predetermined value and the age of the wearer 100 by a predetermined coefficient. The predetermined value may be, for example, 180 or another value. When the WBGT is 31 degrees or higher, the controller 10 may reduce the value of the predetermined coefficient to be smaller than the value thereof in a case in which the WBGT is lower than 31 degrees. The predetermined coefficient when the WBGT is 31 degrees or higher may be, for example, 0.9. The predetermined coefficient when the WBGT is lower than 31 degrees may be, for example, 1.0.

The controller 10 acquires the body temperature or the heart rate of the wearer 100 from the first sensor 20 (step S67).

The controller 10 determines whether the body temperature of the wearer 100 is equal to or higher than the body temperature threshold (step S68). When the body temperature of the wearer 100 is equal to or higher than the body temperature threshold (Yes in step S68), the controller 10 proceeds to step S70.

When the body temperature of the wearer 100 is lower than the body temperature threshold (No in step S68), the controller 10 determines whether the heart rate of the wearer 100 is equal to or higher than the heart rate threshold (step S69). When the body temperature of the wearer 100 is equal to or higher than the body temperature threshold (Yes in step S68), the controller 10 may proceeds to step S70. When the heart rate of the wearer 100 is lower than the heart rate threshold (No in step S69), the controller 10 ends the procedure of the flowchart illustrated in FIG. 9.

When the body temperature of the wearer 100 is equal to or higher than the body temperature threshold (Yes in step S68), or when the heart rate of the wearer 100 is equal to or higher than the heart rate threshold (Yes in step S69), the controller 10 causes the notification interface 40 to notify accordingly (step S70). The controller 10 may execute the processing the same as, or similar to, the processing in step S6 of FIG. 4. After completing step S70, the controller 10 ends the procedure of the flowchart illustrated in FIG. 9.

Even when the moving distance is less than the threshold (Yes in step S63), the controller 10 may proceed to step S64 and correct the values of the acquired temperature and relative humidity for use.

The controller 10 may acquire the oxygen saturation of the wearer 100 in step S67. When the oxygen saturation of the wearer 100 is less than a predetermined threshold, the controller 10 may proceeds to step S70. The predetermined threshold may be, for example, 95%.

The procedure of the flowchart illustrated in FIG. 9 may be used by the server 70 as the procedure to determine whether there is a change in the physical condition of the wearer 100.

According to the helmet 1 of an embodiment, the body temperature of the wearer 100 detected by the body temperature sensor 22 may be corrected based on temperature detected by the ambient temperature sensor 31 or the relative humidity detected by the humidity sensor 32.

The helmet 1 according to an embodiment may be worn by an operator who works at an outdoor site. In this case, the helmet 1 can acquire the biological information of the operator or the environment information of the site in which the operator is located.

The helmet 1 according to an embodiment may be worn by an occupant of a vehicle such as a motorcycle or a bicycle. The helmet 1 according to an embodiment may be worn by a hiker. The helmet 1 according to an embodiment may be worn by a skier or a snowboarder. The helmet 1 according to an embodiment is not limited to these people and may be worn by a variety of other people.

Although the above embodiments have been described based on the figures and the examples, it should be apparent to those skilled in the art that various modifications and alterations can be made without departing from the present disclosure. Accordingly, such modifications and alterations are to be included in the scope of the present disclosure. For example, a function included in each element or each step can be rearranged without logical inconsistency, such that a plurality of elements or steps are combined together, or one element or step is subdivided. It should be understood that, although an apparatus has been mainly described above as the embodiments of the disclosure herein, the embodiments of the present disclosure may also be substantialized by a method that includes a step to be executed by each element of the apparatus. The embodiments of the present disclosure can be implemented by a method to be executed by a processor included in an apparatus, a program, or a storage medium storing the program. Thus, it should be appreciated that such method, program, and storage medium are included in the scope of the disclosure herein.

The descriptions such as “first” and “second” used herein are identifiers for distinguishing the configuration. In the configuration distinguished by the descriptions of “first” and “second”, such numbers can be interchanged. For example, the first sensor and the second sensor can interchange their identifiers: “first” and “second”. The interchange is performed simultaneously. The configuration remains being distinguished after the interchange The identifiers may be omitted. Configurations from which the identifiers are omitted may be distinguished by reference signs. The descriptions of the identifiers “first” and “second” alone should not be used as grounds to define the order of the elements or as grounds to prove the existence of a smaller numbered identifier.

Each of the configurations and processing steps in the embodiments of the present disclosure can be approximately combined with one another.

### REFERENCE SIGNS LIST

1(1a, 1b) helmet

2 cap body

5 hole

10 controller

20 first sensor

21 pulse sensor

22 body temperature sensor

30 second sensor

31 ambient temperature sensor

32 humidity sensor

33 motion sensor

40 notification interface

50 communication interface

60 communication device

70 server

80 network

100 wearer

1. A helmet comprising:
a first sensor for detecting biological information of a wearer;
a second sensor for detecting environment information around the wearer; and
a controller configured to acquire the biological information and the environment information from the first sensor and the second sensor, respectively,
wherein the controller determines a cycle for executing detection by at least one of the first sensor and the second sensor, based on the biological information and the environment information.

2. The helmet according to claim 1, further comprising a communication interface communicably connected to a server via a network,
wherein the controller is configured to:
cause the communication interface to output at least one of the biological information and the environment information to the server via the network, and
cause the communication interface to acquire the cycle for executing detection by at least one of the first sensor and the second sensor from the server via the network.

3. The helmet according to claim 1, wherein the first sensor includes a body temperature sensor.

4. The helmet according to claim 1, wherein the second sensor includes an ambient temperature sensor.

5. The helmet according to claim 4, further comprising a hole penetrating from a wearer side to an exterior,
wherein the ambient temperature sensor is located at the hole.

6. The helmet according to claim 1,
wherein the first sensor includes at least one of an oxygen saturation sensor, a blood flow sensor, and a brain wave sensor.

7. The helmet according to claim 1,
wherein the wearer is an occupant of a vehicle.

8. The helmet according to claim 2,
wherein the first sensor includes a body temperature sensor.

9. The helmet according to claim 2,
wherein the second sensor includes an ambient temperature sensor.

10. The helmet according to claim 3,
wherein the second sensor includes an ambient temperature sensor.

11. The helmet according to claim 8,
wherein the second sensor includes an ambient temperature sensor.

12. The helmet according to claim 9, further comprising a hole penetrating from a wearer side to an exterior,
wherein the ambient temperature sensor is located at the hole.

13. The helmet according to claim 10, further comprising a hole penetrating from a wearer side to an exterior,
wherein the ambient temperature sensor is located at the hole.

14. The helmet according to claim 11, further comprising a hole penetrating from a wearer side to an exterior,
wherein the ambient temperature sensor is located at the hole.

15. The helmet according to claim 2,
wherein the first sensor includes at least one of an oxygen saturation sensor, a blood flow sensor, and a brain wave sensor.

16. The helmet according to claim 3,
wherein the first sensor includes at least one of an oxygen saturation sensor, a blood flow sensor, and a brain wave sensor.

17. The helmet according to claim 4,
wherein the first sensor includes at least one of an oxygen saturation sensor, a blood flow sensor, and a brain wave sensor.

18. The helmet according to claim 2,
wherein the wearer is an occupant of a vehicle.

19. The helmet according to claim 3,
wherein the wearer is an occupant of a vehicle.

20. The helmet according to claim 4,
wherein the wearer is an occupant of a vehicle.
